(19) United States
(12) Patent Application Publication
(10) Pub. No.: US 2025/0316331 A1
(43) Pub. Date: Oct. 9, 2025

(54) IDENTITY BY FUNCTION BASED BLUP METHOD FOR GENOMIC IMPROVEMENT IN ANIMALS

(71) Applicant: INARI AGRICULTURE TECHNOLOGY, INC., Cambridge, MA (US)

(72) Inventors: MALACHY CAMPBELL, Randolph, MA (US); Karl Kremling, Somerville, MA (US); ZACHARY LIPPMAN, Bellmore, NY (US); Ruijuan Li, Belmont, MA (US)

(21) Appl. No.: 18/865,414

(22) PCT Filed: Jan. 20, 2023
(86) PCT No.: PCT/US2023/061007

§ 371 Date: Nov. 13, 2024

Related U.S. Application Data
(60) Provisional application No. 63/267,273, filed on Jan. 28, 2022.
(60) Provisional application No. 63/364,785, filed on May 16, 2022.

(51) Int. Cl. G16B 20/20; G16B 40/00

(52) U.S. Cl. CPC G16B 20/20; G16B 40/00

(57) ABSTRACT

Provided herein are methods for predicting unobserved phenotypes and selecting genetic variant organisms for effective use in genetically improving non-human animal species. Also provided herein are systems for implementing such methods, as well as computer-readable storage media storing instructions for performing such methods.

### CROSS REFERENCE TO RELATED APPLICATIONS

This application claims priority under 35 U.S.C. § 119 to provisional patent applications U.S. Serial Nos. 63/267,273 filed Jan. 28, 2022, and 63/364,785 filed May 16, 2022. The provisional patent applications are herein incorporated by reference in their entirety, including without limitation, the specification, claims, and abstract, as well as any figures, tables, appendices, or drawings thereof.

### BACKGROUND

The traditional phenotype-based breeding and the more recent genomic selection techniques have made significant achievement in improving economically valuable and genetically complex traits (e.g., highly polygenic or controlled by more than 50 genomic loci) in agricultural species, for example, yield performance in maize (Heffner et al., Crop Science, 2009; 49(1):1-12). However, further progress in genetic improvement of such complex traits requires a better prediction and understanding of the underlying genetic variants and identification thereof.

Various efforts have been attempted to address this issue. The use of computational techniques and machine learning methods has aided prediction of the phenotypic consequences and prediction of the phenotypic features using genetic variants. However, current methods and systems are limited in efficiency and accuracy of predicting unobserved phenotypes and selecting genetic variants for effective use in genetically improving agricultural species, as well as in human genetics and medicine.

Accordingly, there is a need for improved methods and systems for identifying organisms with a desired unobserved phenotypic feature. These identified organisms can then be selected and used as candidates for genetic modification, identify candidate sequences as gene editing targets, or identify donors for further breeding to improve desirable traits (e.g., yield performance) in plants and livestock. In addition, these identified organisms can inform treatments or interventions decisions in plant and animal health and human medicine (e.g., nutrition or biological crop protection treatments, or as a target in precision medicine).

In general, the rate of genetic gain over time (Rₜ) is a function of the intensity (i) and accuracy of selection (r), the amount of genetic variation (σ_{g}²) in the population for the trait of interest and the number of cycles of selection that can be performed in a year (y).

$${R}_{t} = \frac{ir {σ}_{g}^{2}}{y}$$

The overarching goal of genomic prediction is to associate phenotypes to genotypes and to predict the genetic merit of often unobserved individuals in a population using genotypic data; thus, facilitating selection without phenotypic evaluation (Meuwissen, Hayes, Goddard, 2001, available on the internet at doi[dot]org/10[dot]1093/genetics/157.4.1819). Moreover, genomic prediction approaches which improve the accuracy of selections can be very valuable in increasing genetic gain. Below, the theory and statistical genomic frameworks underlying genomic prediction is briefly summarized.

Suppose we are given a collection of phenotypic records (y) for n individuals in the population. The goal is to decompose these phenotypes into the true genetic signal (g) and the non-genetic signal (e). The relationship between y and g is given by y=g+e.

If a trait of interest is controlled by 100 genes with the additive effect of gene i represented by aᵢ, then the genetic merit/value for the individuals in the population is given by g=Wa, where W is an n by 100 matrix of allele dosage for each of the genes that control the trait for the n individuals in the population. Thus, the genetic merit is the sum of the effects of all causal genes for a given phenotype. The phenotypic variance for the trait can be similarly decomposed into additive genetic variance and non-additive genetic variance, Vₚ=V_{g}+V_{c}. Similarly, this relationship can be expressed as V_{P}=W′Wσ²_{g}+Iσ²ₑ. If W is centered, then W′W is an n×n covariance matrix that represents the additive genetic relationships between individuals based on the shared alleles at each gene. The cross product of W effectively calculates, for any two individuals, the number of loci in which both individuals are homozygous minus the number of homozygous loci in which they differ (Isik, Holland and Maltecca, 2017). These relationship matrices are analogous to numerator relationship matrices estimated from pedigrees that reflect the expected genetic similarities between sibs, half-sibs and distant relatives, i.e., the probability that alleles are identical by descent (Henderson 1975, VanRaden 2008).

In practice, g and a are unknown and must be predicted from the phenotypic records and genome-wide marker genotypes using one of several statistical genomic frameworks. Dense genotypic data are generated for sites throughout the genome and are often used to compute genomic relationship matrices using similar approaches outlined above. These relationship matrices form the basis for such prediction approaches such as genomic best linear unbiased prediction (GBLUP), which leverage genomic similarities between individuals to predict genetic merit. In practice, relationships are estimated based on shared homozygosity at a single nucleotide level rather than a gene level. Although other whole-regression frameworks utilize marker information differently than GBLUP—specifically by predicting marker effects jointly—these frameworks still rely on site-wise information for prediction (Meuwissen, Hayes, Goddard, 2001, Whittaker and Thompson 2000).

When independent variants can have the same functional effects on a gene (a phenomenon referred to as allelic heterogeneity), site-wise information may inadequately capture the underlying biology of the trait, and predictions may be incomplete and inaccurate. Specifically, with GBLUP, functionally equivalent alleles may not be identical by descent; thus, phenotypic similarities between individuals may not be adequately captured by genomic similarities. Moreover, in regions with allelic heterogeneity, phenotypic variation can be driven by uncorrelated, independent causal variants leading to high error for the predicted marker effects in such regions.

### SUMMARY

Provided herein are methods for predicting unobserved phenotypes and selecting genetic variant organisms for effective use in genetically improving agricultural species, as well as in human genetics and medicine.

In one aspect, provided herein is a method for predicting an desired unobserved phenotype and selecting an organism with improved performance in a population, including: a) providing a population of organisms; b) obtaining genotype data for an organism; c) computing a functional unit dosage matrix (W); d) removing monomorphic functional units; e) computing an identity by function relationship matrix; f) predicting an observed phenotypic feature using a model and; g) utilizing said model to identify an organism having said desired unobserved phenotypic feature.

In some embodiments, the organism is maize, wheat, barley, oat, rice, soybean, oil palm, safflower, sesame, tobacco, flax, cotton, sunflower, pearl millet, foxtail millet, sorghum, canola, cannabis, a vegetable crop, a forage crop, an industrial crop, a woody crop, or a biomass crop. In some embodiments that may be combined with the foregoing, the performance is yield, overall fitness, biomass, photosynthetic efficiency, nutrient use efficiency, heat tolerance, drought tolerance, herbicide tolerance, disease resistance.

In some embodiments, the organism is cattle, sheep, goat, horse, pig, chicken, duck, goose, rabbit, or fish. In some embodiments that may be combined with the foregoing, the growth rate, feed use efficiency, meat yield, meat quality, milk yield, milk quality, egg yield, egg quality, wool yield, or wool quality.

In some embodiments that may be combined with any of the preceding embodiments, the performance is a quantitative trait.

In some embodiments that may be combined with any of the preceding embodiments, the genetic variants are identified by a linkage study. In some embodiments that may be combined with any of the preceding embodiments, the genetic variants are identified by an association study. In some embodiments, the association study is a genome wide association study (GWAS) or a transcriptome-wide association study (TWAS).

In some embodiments that may be combined with any of the preceding embodiments, the statistical model is a linear regression model, a logistic regression model, a ridge regression model, a lasso regression model, an elastic net regression model, a decision tree model, a gradient boosted tree model, a neural network model, or a support vector machine (SVM) model. In some embodiments that may be combined with any of the preceding embodiments, the statistical model comprises a feature based on evolutionary conservation of the genetic variants. In some embodiments, the evolutionary conservation is determined by sequence alignment in a genic or an intergenic region. In some embodiments that may be combined with any of the preceding embodiments, the statistical model comprises a feature based on functional impact of amino acid change of the genetic variants. In some embodiments, the functional impact of amino acid change is weighted according to the blocks substitution matrix (BLOSUM). In some embodiments that may be combined with any of the preceding embodiments, the statistical model comprises a feature based on functional impact of protein conformation and/or stability of the genetic variants. In some embodiments, the functional impact of protein conformation and/or stability is determined by a Monte Carlo search for minimal free energy. In some embodiments, the functional impact of protein conformation and/or stability is predicted by learning a representation of amino acid order from existing proteins in higher dimensional space. In some embodiments that may be combined with any of the preceding embodiments, the statistical model comprises a feature based on adjacency to a selective sweep region of the genetic variants. In some embodiments, the selective sweep region is determined by a decrease of pairwise nucleotide diversity p or linkage disequilibrium relative to the rest of the genome. In some embodiments that may be combined with any of the preceding embodiments, the statistical model comprises a feature based on outlier status of an endophenotype associated with a genetic variant that is physically proximal or proximal within a gene network. In some embodiments that may be combined with any of the preceding embodiments, the feature is a numeric or categorical value associated with a specific allele at a genomic locus.

In certain aspects, the present invention provides an organism with improved performance produced or selected by traditional breeding, market assisted selection, gene editing, and/or transgenesis.

In yet some other aspects, provided herein is a computer-implemented method for predicting an desired unobserved phenotype and selecting an organism with improved performance in a population, including: a) providing a population of organisms; b) obtaining genotype data for an organism; c) computing a functional unit dosage matrix (W); d) removing monomorphic functional units; e) computing an identity by function relationship matrix; f) predicting an observed phenotypic feature using a model and; g) utilizing said model to identify an organism having said desired unobserved phenotypic feature.

In yet some other aspects, provided herein is a computer-readable storage medium storing computer-executable instructions, including: a) instructions for applying a statistical model to a dataset, wherein the dataset comprises a plurality of genetic variants of an organism, and wherein the statistical model comprises one or more initial rules that associate the genetic variants with performance of the organism; and b) instructions for predicting an effect value related to the performance of the organisms. In some embodiments, the computer-readable storage medium further includes instructions for updating the statistical model wherein the statistical model is a linear regression model, a logistic regression model, a ridge regression model, a lasso regression model, an elastic net regression model, a decision tree model, a gradient boosted tree model, a neural network model, or a support vector machine (SVM) model. In some embodiments, the one or more initial rules or the one or more new rules comprise evolutionary conservation, functional impact of amino acid change, functional impact of protein conformation and/or stability, or a combination thereof.

In yet some other aspects, provided herein is a system for predicting unobserved phenotypes and selecting genetic variant organisms for effective use in genetically improving agricultural species, as well as in human genetics and medicine, including: a) a computer-readable storage medium storing a database comprising a plurality of genetic variants of the organism; b) a computer-readable storage medium storing computer-executable instructions, including: i) instructions for applying a statistical model to the dataset, wherein the statistical model comprises one or more initial rules that associate the genetic variants with performance of the organism; and ii) instructions for calculating an effect value related to the performance of the organism for each of the genetic variants; and c) a processor configured to execute the computer-executable instructions stored in the computer-readable storage medium. In some embodiments, the computer-readable storage medium further includes instructions for updating the statistical model with one or more new rules, wherein the statistical model is a linear regression model, a logistic regression model, a ridge regression model, a lasso regression model, an elastic net regression model, a decision tree model, a gradient boosted tree model, a neural network model, or a support vector machine (SVM) model. In some embodiments, the one or more initial rules or the one or more new rules comprise evolutionary conservation, functional impact of amino acid change, functional impact of protein conformation and/or stability, or a combination thereof.

In yet some other aspects, provided herein is a method for selecting one or more of the genetic variants from a population of organisms. In some embodiments, the statistical model comprises calculating the effect of a genetic variant on the biological function of a protein. In some embodiments, the organism is cattle, sheep, goat, horse, pig, chicken, duck, goose, rabbit, or fish. In some embodiments the performance of the organism is growth rate, feed use efficiency, meat yield, meat quality, milk yield, milk quality, egg yield, egg quality, wool yield, or wool quality.

In some embodiments, the genetic variants comprise a deleterious allele that confers or correlates with a negative effect to the performance of the organism. In some embodiments, the deleterious allele is overexpressed or underexpressed in the organism in comparison to a control organism. In some embodiments, the genetic variants are homozygous or heterozygous in the organism. In some embodiments, the genetic variants comprise a deleterious allele that is homozygous in the organism. In some embodiments, the prioritized genetic variants comprise a target for gene editing. In some embodiments, the prioritized genetic variants comprise a deleterious allele homozygous in the organism that is used as a target for gene editing.

### DESCRIPTION OF THE FIGURES

Figures IA-C. Examples of collapsing functionally-equivalent loss of function (LoF) LoF variants at a gene level.

FIGS. 2A-B. Examples of collapsing functionally-equivalent variants at a codon level.

FIGS. 3A-C. Examples of collapsing functionally-equivalent LoF variants at a pathway level.

FIGS. 4A-B. Evaluating predictive ability of two-kernel genomic prediction method.

### DETAILED DESCRIPTION

The phrase “allelic variant” and/or “variant” as used herein refers to a polynucleotide or polypeptide sequence variant that occurs in a different strain, variety, or isolate of a given organism.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the terms “comprise,” comprises, “comprising,” “include,” “includes,” and “including” can be interchanged and are to be construed as at least having the features to which they refer while not excluding any additional unspecified features.

As used herein, the phrase “target gene” can refer to either a gene located in the genome that is to be modified by gene editing molecules provided in a system, method, composition and/or eukaryotic cell provided herein. Embodiments of target genes include (protein-)coding sequence, non-coding sequence, and combinations of coding and non-coding sequences. Modifications of a target gene include nucleotide substitutions, insertions, and/or deletions in one or more elements of a gene that include a transcriptional enhancer or promoter, a 5′ or 3′ untranslated region, a mature or precursor RNA coding sequence, an intron, a splice donor and/or acceptor, a protein coding sequence, a polyadenylation site, and/or a transcriptional terminator. In certain embodiments, all copies or all alleles of a given target gene in an animal cell are modified to provide homozygosity of the modified target gene in the animal cell. In embodiments, where a desired trait is conferred by a loss-of-function mutation that is introduced into the target gene by gene editing, an animal cell, population of animal cells, embryo, or animal is homozygous for a modified target gene with the loss-of-function mutation. In other embodiments, only a subset of the copies or alleles of a given target gene are modified to provide heterozygosity of the modified target gene in the animal cell. In certain embodiments where a desired trait is conferred by a dominant mutation that is introduced into the target gene by gene editing, an animal cell, population of animal cells, embryo, or animal is heterozygous for a modified target gene with the dominant mutation. Traits imparted by such modifications to certain target genes may include improved yield, resistance to disease, bacterial pathogens, and/or fungal infections, stress tolerance (e.g., cold and/or heat tolerance), protein quantity and/or quality, fat quantity and/or quality, and the like, all in comparison to a control organism that lacks the modification. The animal having a genome modified by gene editing molecules provided in a system, method, composition and/or animal cell provided herein differs from an animal having a genome modified by traditional breeding (i.e., crossing of a male organism and a female organism), where unwanted and random exchange of genomic regions as well as random mitotically or meiotically generated genetic and epigenetic changes in the genome typically occurs and are then found in the progeny population. Thus, in embodiments of the animal (or animal cell) with a modified genome, the modified genome is more than 99.9% identical to the original (unmodified) genome. In some embodiments, the modified genome is devoid of random mitotically or meiotically generated genetic or epigenetic changes relative to the original (unmodified) genome. In embodiments, the modified genome includes a difference of epigenetic changes in less than 0.01% of the genome relative to the original (unmodified) genome. In embodiments, the modified genome includes: (a) a difference of DNA methylation in less than 0.01% of the genome, relative to the original (unmodified) genome; or (b) a difference of DNA methylation in less than 0.005% of the genome, relative to the original (unmodified) genome; or (c) a difference of DNA methylation in less than 0.001% of the genome, relative to the original (unmodified) genome. In embodiments, the gene of interest is located on a chromosome in the animal cell, and the modified genome includes: (a) a difference of DNA methylation in less than 0.01% of the portion of the genome that is contained within the chromosome containing the gene of interest, relative to the original (unmodified) genome; or (b) a difference of DNA methylation in less than 0.005% of the portion of the genome that is contained within the chromosome containing the gene of interest, relative to the original (unmodified) genome; or (c) a difference of DNA methylation in less than 0.001% of the portion of the genome that is contained within the chromosome containing the gene of interest, relative to the original (unmodified) genome. In embodiments, the modified genome has not more unintended changes in comparison to the original (unmodified) genome than 1×10⁸ mutations per base pair per replication. In certain embodiments, the modified genome has not more unintended changes than would occur at the natural mutation rate. Natural mutation rates can be determined empirically or are as described in the literature (Lynch, M., 2010; Clark et al., 2005).

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown but are to be accorded the scope consistent with the claims.

Genetic variants refer to the alternate sequences of DNA at a specific region of the genome between organisms, or the alternate amino acid sequences encoded thereby, which serve as the source and targets for genetic improvement of organisms. However, the number of genetic variants for a given genome can be enormous, and the effect of a genetic variant can be either neutral, favorable, or deleterious to the fitness and performance of an organism. Therefore, to achieve efficient and effective genetic improvement of an organism, genetic variants need to be assessed for their effects such that subsequent breeding effort can be prioritized in selecting for or against such variants or modifying thereof.

Provided herein are methods for predicting the unobserved phenotype of genetic variants for use in genetically improving organisms and in animal genetics and medicine. Also provided herein are systems for implementing such methods, as well as computer-readable storage media storing instructions for performing such methods.

Accordingly, in one aspect, provided herein is a method for predicting an desired unobserved phenotype and selecting an organism with improved performance in a population, including: a) providing a population of organisms; b) obtaining genotype data for an organism; c) computing a functional unit dosage matrix (W); d) removing monomorphic functional units; e) computing an identity by function relationship matrix; f) predicting an observed phenotypic feature using a model and; g) utilizing said model to identify an organism having said desired unobserved phenotypic feature.

As used herein, the terms “genetic variant” and/or “variant” refer to a nucleotide or polypeptide sequence that differ from a reference sequence for a given region. For example, a genetic variant may comprise a deletion, substitution, or insertion of one or more nucleotides or amino acids encoded thereof. When the reference sequence refers to a normal or wild-type sequence, a genetic variant may also be referred to as a “mutation” and an organism having such mutation as a “mutant.” When it is used in the context of an alternative form of a sequence, especially that of a gene in a population, a genetic variant may also be referred to as an “allele.” Accordingly, in some embodiments, the genetic variant of the present disclosure is allele. In some embodiments, the genetic variant is a mutation.

Various types of genetic variants may be used with the methods of the present disclosure, which include, for example, frameshift, stop gained, start lost, splice acceptor, splice donor, stop lost, inframe indel, missense, splice region, synonymous, and copy number variants. Non-limiting types of copy number variants include deletions and duplications.

The genetic variants in the present disclosure may be provided by comparing different sequences at a given region. Methods and techniques of sequencing and sequence alignment are known in the art. See e.g., Adams et al., eds. Automated DNA sequencing and analysis. Elsevier, 2012, França et al., Quarterly reviews of biophysics, 35(2), 169-200, and Rosenberg, M. S. ed., 2009. Sequence alignment: methods, models, concepts, and strategies. Univ of California Press.

In some embodiments, the genetic variants of the present invention are those that exhibit epistasis. As used herein, the term “epistasis” (also known as “epistatic interaction” or “epistatic relationship”) refers to an interaction between variants of within or between genetic sequences, including, for example, genetic variants, where the presence of one genetic variant has an effect conditional on the presence of one or more additional genetic variants. Epistasis occurs both within and between molecules. Epistatic sequences may refer to alleles of a gene, genetic variants (e.g., mutations) of a gene, or sequences (e.g., genes, genetic variants) within a gene network or within a genome. Epistasis may be of various types, including, for example, dominant, recessive, complementary, compensatory, and polymeric interaction. A compensatory secondary genetic variant, for example, exhibits a compensatory epistatic interaction with a primary genetic variant. As used herein, a “compensatory” or “compensating” effect refers to a counteracting, offsetting, mitigating, and/or opposing effect. For example, relevant to a primary genetic variant, a “compensatory” or “compensating” secondary genetic variant would have a “compensatory effect” that counteracts, offsets, mitigates, and/or opposes the effect of the primary genetic variant. A compensatory secondary genetic variant may be within the same gene or gene product (e.g., polypeptide) as the primary genetic variant, i.e., a cis-acting compensatory genetic variant. A compensatory secondary genetic variant may be in a different gene or gene product (e.g., polypeptide) as the primary genetic variant, i.e., a trans-acting compensatory genetic variant. In some embodiments, the trans-acting compensatory genetic variant is within the same gene network as the primary genetic variant.

In some embodiments, the effect of a genetic variant may be represented in a numerical or mathematical form, such as an effect score. The terms “effect score” and “fitness score” refer to a representation of the effect of a variant relative to a reference or wild-type sequence. The representation may be interpretable to humans and/or machines.

The effect of a genetic variant may also refer to a value or score from a statistical model or test, including for example, a P value from a likelihood ratio test (Knudsen, B. and Miyamoto, M. M., 2001. A likelihood ratio test for evolutionary rate shifts and functional divergence among proteins. Proceedings of the National Academy of Sciences, 98(25), pp. 14512-14517), a SIFT score (Ng, P. C. and Henikoff, S., 2003. SIFT: Predicting amino acid changes that affect protein function. Nucleic acids research, 31(13), pp. 3812-3814), and a PROVEAN score (Choi, Y., Sims, G. E., Murphy, S., Miller, J. R. and Chan, A. P., 2012. Predicting the functional effect of amino acid substitutions and indels. PloS one, 7(10), p.e46688). In some embodiments, SIFT is performed with proteins having at least 80%, at least 85%, at least 90% or at least 95% identity. In some embodiments, a genetic variant is deleterious if the SIFT score is less than 0.1, less than 0.05, or less than 0.01.

Accordingly, in one aspect, provided herein is a method for predicting a desired unobserved phenotype and selecting an organism with improved performance in a population, including: a) providing a population of organisms; b) obtaining genotype data for an organism; c) computing a functional unit dosage matrix (W); d) removing monomorphic functional units; e) computing an identity by function relationship matrix; f) predicting an observed phenotypic feature using a model and; g) utilizing said model to identify an organism having said desired unobserved phenotypic feature.

The organism of the present invention may be any organism that is of economic and/or scientific value to humans. In some embodiments, the organism is an animal. In some embodiments, the organism is cattle, sheep, goat, horse, pig, chicken, duck, goose, rabbit, or fish. In some embodiments, the organism is an alga, such as spirulina.

The performance of the present invention may be any phenotype, quality, or trait of the organism. For instance, in some embodiments wherein the organism is an animal, the performance may be growth rate, feed use efficiency, meat yield, meat quality, milk yield, milk quality, egg yield, egg quality, wool yield, or wool quality. A list of exemplary phenotypes of interested is provided below in Table 1.

TABLE 1
List of exemplary traits/pathogens.

| Trait | Trait Class |
|---|---|
| Meat yield | Yield |
| Meat quality | Yield |
| Milk yield | Yield |
| Milk quality | Yield |
| Egg yield | Yield |
| Egg quality | Yield |
| Wool yield | Yield |
| Wool quality | Yield |
| Growth rate | Metabolism |
| Feed use efficiency | Metabolism |
| Fertility | Metabolism |
| Avian influenza | Disease resistance |
| Virulent Newcastle Disease | Disease resistance |
| Infectious Salmon Anemia Virus (ISAV) | Disease resistance |
| Brucellosis | Disease resistance |
| Bovine Spongiform Encephalopathy | Disease resistance |
| (BSE) | |
| Chronic Wasting Disease | Disease resistance |
| African Horse Sickness | Disease resistance |
| Contagious Equine Metritis | Disease resistance |
| Eastern/Western/Venezuela Equine | Disease resistance |
| Encephalitis | |
| Equine Herpes Virus | Disease resistance |
| Equine Viral Arteritis | Disease resistance |
| Equine Infectious Anemia | Disease resistance |
| Vesicular Stomatitis | Disease resistance |
| West Nile Virus | Disease resistance |
| Equine Piroplasmosis | Disease resistance |
| Scrapie | Disease resistance |
| Classical Swine Fever | Disease resistance |
| Pseudorabies | Disease resistance |
| Rabies | Disease resistance |
| Salmonella | Disease resistance |
| Coccidiosis | Disease resistance |
| Foot and mouth disease (FMD) | Disease resistance |
| Porcine reproductive and respiratory | Disease resistance |
| syndrome (PRRS) | |
| E. Coli | Disease resistance |
| West Nile Virus | Disease resistance |
| Mastitis | Disease resistance |
| African Swine Fever | Disease resistance |

In some embodiments, the identified organisms and genetic variants therein of the present disclosure may be used as targets in precision medicine. As used herein, the terms “personalized medicine,” “individualized medicine,” and “precision medicine” refer to the tailoring of medical procedures to the individual characteristics of each organism, based on the organism's unique molecular and genetic profile that make the patient predisposed or susceptible to certain diseases. A medical procedure may be prognosis, diagnosis, treatment, intervention, or prevention.

The genetic variants in the present invention may be provided by comparing sequences between genomes. Methods and techniques of sequencing and sequence alignment are known in the art. See e.g., Adams et al., eds. Automated DNA sequencing and analysis. Elsevier, 2012, França et al., Quarterly reviews of biophysics, 35(2), 169-200, and Rosenberg, M. S. ed., 2009. Sequence alignment: methods, models, concepts, and strategies. Univ of California Press. In certain variations, the genetic variants that are associated with performance of the organism are provided. In some embodiments, the genetic variants may be identified by a linkage study. In some embodiments, the genetic variants may be identified by an association study. In some embodiments, the association study is a genome wide association study (GWAS) or a transcriptome-wide association study (TWAS).

Statistical models and machine learning have been used in predicting effects of genetic variants in plant and animal breeding and human medicine. Methods and techniques of statistical modeling are known in the art. See e.g., Varshney, et al. Trends in biotechnology, 2009; 27(9), 522-530, Cardoso et al. Front Bioeng Biotechnol. 2015; 3: 13, and Ho et al. Frontiers in Genetics, 2019; 10. The statistical model of the present invention may be any statistical model that associates the genetic variants with the performance of the organism. Accordingly, in some embodiments, the statistical model may be a linear regression model, a logistic regression model, a ridge regression model, a lasso regression model, an elastic net regression model, a decision tree model, a gradient boosted tree model, a neural network model, or a support vector machine (SVM) model.

By way of example, putatively deleterious alleles and their impacts on phenotypic performance may be predicted using sequential natural language deep learning models. As used herein, the term “language model,” which may refer to either a “sequential language model” or a “masked language model” refers to a machine learning method that interprets, predicts, and/or generates sequential data. At a high level, a sequential language model takes in a sequence of inputs, examines each element of the sequence, and predicts the next element of the sequence. Similarly, a masked language model takes in a sequence of inputs. a random subset of which have their ground truth masked or obscured from the perspective of the model and predicts those masked elements. In some embodiments, the language model is a mathematical representation of the frequency and order with which specific monomeric units or gaps occur in a set of polymers. e.g., amino acid residues in a polypeptide sequence. The mathematical representation can include a probability of a given monomer occurring at a position in the sequence. In some embodiments, the language model predicts what specific monomer comes next in a sequence of different monomers—a process known as “next token prediction.” In some embodiments, the language model predicts what specific monomer should fill in a missing space in a sequence of different monomers—a process known as “masked token prediction.” A probability of a given monomer occurring at a position in the sequence model can be independent of other positions or can depend on the occupancy at any or all other positions in the sequence model. An example of a position independent model is a Hidden Markov Model. In some embodiments, the language model is configured to output a set of semantic features. These models uniquely permit the prediction of an allele's impact when it is present in combination with secondary or in higher order combination with other putatively deleterious alleles which may in fact be compensatory for the impact of the focal mutation, rendering it non deleterious. The correct prediction of these compensations through the use of sequential natural language models reduces false positive and false negative misprioritization of alleles which in turn leads to loss rather than gain of yield performance after editing such a false positive nomination of the deleterious allele.

The genetic variants of the organism in the present invention may be assessed, weighted, or prioritized by a statistical model based on one or more criteria. Examples of the criteria include, but are not limited to, evolutionary conservation (See e.g. Chun and Fay (2009) Genome Res. 19: 1553-1561 and Rodgers-Melnick et al (2015) PNAS 112: 3823-3828), functional impact of amino acid change (See e.g. Ng et al (2003) NAR 31:3812-3814 and Adzhubei et al (2010) Nat Methods 7:248-249), and functional impact of protein conformation and/or stability (See e.g. Rosetta, a computational protein design platform from Cyrus Bio Inc.). In some embodiments, the evolutionary conservation is determined by sequence alignment in a genic or an intergenic region. In some embodiments, the functional impact of amino acid change is weighted according to the blocks substitution matrix (BLOSUM). In some embodiments, the functional impact of protein conformation and/or stability is determined by a Monte Carlo search for minimal free energy. In some embodiments, the functional impact of protein conformation and/or stability is predicted by learning a representation of amino acid order from existing proteins in higher dimensional space. In some embodiments that may be combined with any of the preceding embodiments, the feature is a numeric or categorical value associated with a specific allele at a genomic locus.

In some embodiments, the alteration/perturbation of the genetic variants is achieved by genome editing. As used herein, the term “genome editing” or “gene editing” refers to the process of altering the target genomic DNA sequence by inserting, replacing, or removing one or more nucleotides. Genome editing may be accomplished by using nucleases, which create specific double-strand breaks (DSBs) at desired locations in the genome and harness the cell's endogenous mechanisms to repair the induced break by homology-directed repair (HDR) (e.g., homologous recombination) or by non-homologous end joining (NHEJ). Any suitable nuclease may be introduced into a cell to induce genome editing of a target DNA sequence including, but not limited to, clustered regularly interspersed short palindromic repeats (CRISPR)-associated protein (Cas, e.g. Cas9 and Cas12a) nucleases, zinc finger nucleases (ZFNs, e.g. Fokl), transcription activator-like effector nucleases (TAFENs, e.g. TAFEs), meganucleases, and variants thereof (Shukla et al. (2009) Nature 459: 437-441; Townsend et al (2009) Nature 459: 442-445). Accordingly, in some embodiments of the present invention, the genome editing is achieved by a clustered regularly interspersed short palindromic repeats (CRISPR) system, a transcription activator-like effector nuclease (TAFEN) system, or a zinc finger nuclease (ZFN) system.

In some embodiments, the type of genome editing is base editing. As used herein, the term “base editing” refers to a base mutation (substitution, deletion or addition) that causes point mutations in a target site within a target gene, with a few bases (one or two). Various base editors are known in the art and may have various approximate editing windows. See e.g., Rees, H. A. and Liu. D. R., 2018. Base editing: precision chemistry on the genome and transcriptome of living cells. Nature reviews genetics, 19(12), pp. 770-788; Molla, K. A. and Yang, Y., 2019. CRISPR/Cas-mediated base editing: technical considerations and practical applications. Trends in biotechnology, 37(10), pp. 1121-1142; and Mishra, R., Joshi, R. K. and Zhao, K., 2020. Base editing in crops: current advances, limitations and future implications. Plant Biotechnology Journal, 18(1), pp. 20-31. Accordingly, in some embodiments, the editing window is from 5-10 bp. In some embodiments, the editing window is from 5-15 bp. In some embodiments, the editing window is from 5-20 bp. In some embodiments, the editing window is from 5-25 bp. In some embodiments, the editing window is from 5-30 bp. In some embodiments, the editing window is from 5-35 bp. In some embodiments, the editing window is from 5-40 bp. In some embodiments, the editing window is from 5-45 bp. In some embodiments, the editing window is from 5-50 bp. In some embodiments, the editing window is from 10-20 bp. In some embodiments, the editing window is from 10-30 bp. In some embodiments, the editing window is from 10-40 bp. In some embodiments, the editing window is from 10-50 bp.

In yet some other embodiments, the alteration/perturbation of the genetic variants is achieved by creation of novel haplotype combinations from genetic recombination during meiosis in the course of breeding with the aim of increasing the numbers of favorable alleles which are stacked together and inherited together as part of a haplotype. The presence of individual mutations and their abundance can be assessed by genotyping.

In some aspects of the present invention, the method for selecting an organism with improved performance in a population may be used for genomic selection. In some aspects of the present invention, the prioritized genetic variants may be used for genomic selection. Genomic selection (GS) estimates marker effects across the whole genome on the target population based on a prediction model developed in the training population. Methods and techniques of GS is known in the art. See e.g., Jannink, et al. Briefings in functional genomics, 2010: 9(2), 166-177, Goddard, et al. Journal of Animal breeding and Genetics 2007:124 (6), 323-330, and Desta and Ortiz.

In certain aspects, provided herein is an organism with improved performance produced or selected by any one of the methods disclosed in the present invention.

In certain other aspects, provided herein is a computer-implemented method for predicting an desired unobserved phenotype and selecting an organism with improved performance in a population, including: a) providing a population of organisms; b) obtaining genotype data for an organism; c) computing a functional unit dosage matrix (W); d) removing monomorphic functional units; e) computing an identity by function relationship matrix; f) predicting an observed phenotypic feature using a model and; g) utilizing said model to identify an organism having said desired unobserved phenotypic feature.

In yet certain other aspects, provided herein is a computer-readable storage medium storing computer-executable instructions, including: a) instructions for applying a statistical model to a dataset, wherein the dataset comprises a plurality of genetic variants of an organism, and wherein the statistical model comprises one or more initial rules that associate the genetic variants with performance of the organism; and b) instructions for predicting an effect value related to the performance of the organisms. In some embodiments, the computer-readable storage medium further includes instructions for updating the statistical model wherein the statistical model is a linear regression model, a logistic regression model, a ridge regression model, a lasso regression model, an elastic net regression model, a decision tree model, a gradient boosted tree model, a neural network model, or a support vector machine (SVM) model. In some embodiments, the computer-readable storage medium further includes instructions for updating the statistical model. In some embodiments, the computer-readable storage medium is a solid-state device, a hard disk, a CD-ROM, or other non-volatile computer-readable storage medium.

In still certain other aspects, provided herein is a system (e.g., a computer system) for assessing genetic variants for use in genetic improvement of an organism, including: a) a computer-readable storage medium storing a database comprising a plurality of genetic variants of the organism; b) a computer-readable storage medium storing computer-executable instructions, including: a) instructions for applying a statistical model to a dataset, wherein the dataset comprises a plurality of genetic variants of an organism, and wherein the statistical model comprises one or more initial rules that associate the genetic variants with performance of the organism; and b) instructions for predicting an effect value related to the performance of the organisms. In some embodiments, the one or more initial rules or the one or more new rules comprise evolutionary conservation, functional impact of amino acid change, functional impact of protein conformation and/or stability, or a combination thereof. In some embodiments, the system may be a server computer, a client computer, a personal computer, a user device, a tablet PC, a laptop computer, a personal digital assistant, a cellular telephone, or any machine capable of executing a set of instructions, sequential or otherwise, that specify actions to be taken by that machine. In some embodiments, the system may further include keyboard and pointing devices, touch devices, display devices, and network devices.

In some embodiments that may be combined with any of the preceding embodiments, the statistical model is a linear regression model, a logistic regression model, a ridge regression model, a lasso regression model, an elastic net regression model, a decision tree model, a gradient boosted tree model, a neural network model, or a support vector machine (SVM) model.

In some embodiments, aggregating functionally equivalent variants into a single predictor may be an effective strategy to account for allelic heterogeneity, and help increase prediction accuracies and reduce error for traits that are influenced by allelic heterogeneity.

Variants which may be collapsed on the basis of functional equivalence can include, but are not limited to, those which destroy the function of a protein such as through a premature stop codon, out of frame indel, retention of an intron, skipping of an exon, and a point mutation that leads to a severe missense mutation that destroys a conserved protein region. They may also include multiple different mutations of the same class and have similar impacts which simply occur at different positions in the protein, or different positions within a codon. By way of non-limiting example, they may also include SNPs in different positions leading to the same amino acid substitution. Additionally, mutations that are functionally equivalent and would thus be collapsed may include those which lead to a gain of function such as enhanced binding of a substrate by a protein as opposed to a loss of function. Additionally, variants occurring in different proteins in a pathway which have equivalent impacts could be collapsed with a resulting gain in prediction accuracy.

Below, a framework is outlined in which functionally equivalent variants in the same gene or in different genes participating in a common biological process are collapsed to estimate gene-wise or pathway-wise dosages and these data are used in conjunction with conventional genomic relationship matrices to predict the genetic merit of individuals for a trait of interest. In other words, we outline an approach to define genomic relationships based on identity by function rather than identity by state.

In FIG. 1, an example of collapsing functionally equivalent LoF variants at the gene level is presented. FIG. 1A represents the distribution of four SNP markers in hypothetical genomic region. FIG. 1B represents the allele dosages for the four SNP markers and nine individuals are depicted by the matrix W^{SNP} and the incidence matrix, L, that assigns LoF SNPs to genes, as well as the computation of the LoF allele dosage matrix. Non-zero elements indicate the number of copies of the LoF allele. FIG. 1C represents the genomic relationship matrices computed from SNP allele dosages (G^{SNP}) and LoF allele dosages (G^{LoF}). Two cases are highlighted in which the genetic relatedness changes base on whether identity by descent or identity by function is used to define relationships.

The concept of aggregating functionally equivalent alleles has been used for genomic inference (i.e., genome-wide association mapping) and, to a lesser extent, genomic prediction. Sverdlov and Thompson ((2013) Theor. Pop. Biology, 88:57-67; doi.org/10.1016/j.tpb.2013.06.004) describe a framework to estimate the phenotypic and genomic relationships between two individuals in a population that does not rely on IBD relationships—relatedness based on quantifying sequence similarity—but rather, identity by function. This work is motivated by the idea that a gene sequence that differs by one non-synonymous, possibly protein-altering substitution between a pair of individuals will be no more functionally similar that one that differs by multiple non-synonymous substitutions. The authors define a locus as a genic region and an allele as a haplotype for that genic region; thus, each locus can have more than two alleles. For a given locus the relationship between two diploid individuals can be described using one of five possible functional states: both individuals are homozygous for the same allele, heterozygous for the same alleles, share three alleles, share two alleles, or do not share any alleles. The two individuals would be functionally identical in first two instances and their correlation would be one, functionally similar in the third and fourth instances and their correlation would be less than one, and functionally independent in the final instance. When considering relationships at multiple loci, the relationship for any two individuals is simply the sum of functional identity states weighted across loci. Weights can either be locus-specific or population-based. Sverdlov ((2014, available at the internet site digital[dot]lib[dot]Washington[dot]edu/researchworks/bitstream/handle/1773/27121/Sverdlov_washington_0250E_13354.pdf?sequence=1&isAllowed=y) describes an approach to compute a functional genomic relationship matrix which shares the same properties as the realized relationship matrices described by VanRaden (2008). The covariance based on functional identity states across loci is scaled by the expected variance computed from allele frequencies.

The approach of the instant invention shares a key motivation with other approaches in attempting to define relationships based on functional similarities rather than pedigree or sequence similarities, but the instant invention addresses this in a novel manner. The instant invention utilizes a two-kernel genomic BLUP approach, where one kernel is a genomic relationship matrix that defines relationships based on the similarities in functional units between any two individuals in the population, and we assume a single variance component for each of the two kernels. While our approach can be solved using REML or MCMC approaches, the framework outlined by Sverdlov (2014) requires more complex optimization algorithms to cope with the large number of variance components that need to be estimated.

Aside from the analytical difference between the two approaches, our approach leverages prior biological knowledge generated from empirical studies or bioinformatic predictions to aggregate information at various functional levels, e.g., intra-gene level (in the case of independent variants that equally impact a codon or gene function) or inter-gene level (variants that equally impact a pathway or biological process). Moreover, we assume each functional unit can only have two alleles. The approach outlined by Sverdlov (2014) and Sverdlov and Thompson (2013) defines the gene as the functional unit.

To properly account for allelic heterogeneity in genomic prediction, functionally equivalent variants must be aggregated for each functional unit and dosages at each functional unit computed. The term functional unit can describe a codon within a protein, a gene, or a pathway as multiple mutations can result in the same amino acid substitutions, elimination of gene/protein function, or equally perturb a pathway. The examples below describe the approach in terms of genes, but the framework can be applied more generally to any of the functional units described above.

Suppose we genotype a population of nine diploid individuals at a genomic region that spans two genes with four SNP markers that each type a LoF variant. Two LoF markers are typed for each gene. The distribution of variants across this hypothesis genomic region is shown in FIG. 1A. The computation of LoF dosages requires genotypes at each SNP (W^{SNP}; FIG. 1B), as well as an incidence matrix that assigns LoF SNP markers to genes (L; FIG. 1B). The cross product of W^{SNP} and L gives a matrix X^{LOF} where the entry x^{LOF}_{i,j} is the number of LoF alleles for gene j for individual i. Since in this example we assume the SNPs are unphased and LoF alleles are in repulsion, there can be some cases where an element is greater than two. In such cases, these entries are replaced with two.

Once the LoF dosages have been computed, genomic relationships can be defined using any standard frameworks (VanRanden 2008, Yang et al 2010). The relationship matrices pictured in FIG. 1C were computed using VanRaden's second definition, G^{LoF}=(W^{LoF}_{sc}W^{LoF}_{sc}′)/m, where W^{LoF}_{sc} is the scaled and centered matrix of LoF allele dosages. The two highlighted elements in FIG. 1C emphasize the importance of aggregating functionally equivalent variants when defining genomic relationships. Individuals 3 and 7 are homozygous for two functionally equivalent SNPs; however, when SNP-based allele dosages are used to compute relationships these individuals show a negative covariance indicating that their haplotypes are not shared, while relationships defined from LoF dosages show that these two individuals share a functionally equivalent haplotype. Similar patterns can be seen for individuals 2 and 8.

Given that relationships defined from LoF dosage are computed using standard statistical genomic frameworks, we can leverage prediction frameworks such as genomic best linear unbiased prediction to predict the genetic merit of individuals from LoF allele dosages, or more accurately, genomic relationships defined from LoF allele dosages. The standard GBLUP model is given by y=Xb+Zu^{SNP}+e, where X is an incidence matrix that assigns the n observations in y to fixed effects, b is the vector of fixed effect estimates, Z is an incidence matrix that assigns observations to random additive genetic values contained in u, and e is a vector of residuals. We assume that the random effects follow a Gaussian distribution (u^{SNP}˜N(0,σ²_{g}^{SNP}G^{SNP}); e˜N(0, σ²ₑ I)). In practice, we introduce a second additive genetic effect (u^{LoF}) that captures the portion of phenotypic variation that is due to allelic heterogeneity, and we assume u^{LoF}˜N(0, σ²_{g}^{LoF} G^{LoF}). Thus, with this two-kernel framework the additive genetic values for each individual are the sum of the two random additive genetic effects and should account for variation in the phenotype explained by IBD genomic relationships and relatedness due to functionally equivalent haplotypes/alleles. Parameters can be estimated using restricted maximum likelihood or Markov chain Monte Carlo approaches.

The fixed effects model is only applicable when the number of parameters to estimate in the model is one less than the number of individuals in the population. In practice the number of parameters should be far less. This model assumes all the genetic factors contributing to the phenotype is known and can be estimated.

Alternatively, LoF allele dosages can be used directly for prediction using a whole genome prediction model. As above, two random additive genetic effects are used in the model—one set for SNP allele dosages and one for LoF allele dosages—and allele substitution effects are predicted jointly for each set. The advantage of these approaches is that it allows individual locus effects to be predicted and studied, which enables inference about individual locus effects. Moreover, using Bayesian methods the allele substitution effects can be drawn from a variety of densities which may better match the expected genetic architecture of the trait.

The models above assume an additive mode of inheritance; however, many studies have shown that other genetic mechanisms, e.g., dominance and epistasis, also explain a portion of phenotypic variation (Huang et al 2012; Forsberg et al 2017; Mackay 2014; Technow et al 2012; Zhao et al 2013). Moreover, many existing methods are available to define epistatic or dominance relationship matrices from allele dosages; thus, the frameworks presented above can easily be tailored to model epistatic and/or dominance relationships based on identity by function and are a current research interest to the inventors (Nishio and Satoh 2014; Su et al 2012). In addition, we are currently exploring a variety of machine learning approaches to identify functionally equivalent alleles to better define the L matrix. Finally, our current implementation assumes genotypes are unphased and any functionally equivalent alleles are in repulsion. Methods are currently being explored to accommodate phased genotype calls and compute strand-aware dosages for each gene.

### Mitigating the Loss of Genetic Diversity in Breeding Programs.

In many breeding programs that routinely utilize IBD-based prediction/selection, breeders will make selections that increase genetic gain, advancing the most elite lines which are later used in crosses (Meuwissen 1997, Heffner et al 2009, Jannink 2010). With relationship-based approaches, such as GBLUP, the most desirable individuals in a population (based on estimated genetic breeding values, GEBVs) will be related; thus, as breeders continually select the most elite individuals to advance the population, coancestry in the breeding population increases and the overall genetic diversity in the population is reduced. In the approach described above, individuals with different haplotypes can contribute to the same predictor (gene-level LoF dosage); therefore, the most elite individuals based on GEBVs computed from gene-level LoF dosages may not necessarily be related. Selections based on these predictions should not erode genetic diversity as much as selections based on GBLUP GEBVs.

### EMBODIMENTS

Various embodiments of the systems and methods provided herein are included in the following non-limiting list of embodiments.

1. A method for identifying a non-human animal organism with a desired unobserved phenotypic feature, said method comprising:
(a) obtaining genotype data for an organism against a plurality of markers (m);
(b) extracting functionally equivalent alleles for each functional unit;
(c) computing a functional unit dosage matrix (W);
(d) removing monomorphic functional units;
(e) computing an identity by function relationship matrix;
(f) predicting said unobserved phenotypic feature using a best linear unbiased prediction (BLUP) model; and
(g) utilizing said model to identify a non-human animal organism having said desired unobserved phenotypic feature.
   2. The method of embodiment 1, wherein said BLUP model is a two-kernel BLUP model.
   3. The method of embodiment 1, wherein said BLUP model comprises the equation set forth in Equation (1):

$$y = Xb + {Zu}_{GRM} + {Zu}_{FE} + e , \qquad ( 1 )$$

wherein:
   (i) y is the phenotypic response variable,
   (ii) X is an incidence matrix that assigns the n observations in y to fixed effects,
   (iii) b is the vector of fixed effect estimates,
   (iv) Z is an incidence matrix that assigns observations to random additive genetic values contained in u_{GRM} and u_{FE},
   (v) u_{GRM} is a vector of additive genetic values modeled by identity by descent relationships,
   (vi) u_{FE} is a vector of additive genetic values modeled by identity by function relationships (sharing of functionally equivalent functional units), and;
   (vii) e is a vector of residuals.
      4. The method of embodiment 3, wherein the random effects follow a Gaussian distribution.
      5. The method of embodiment 1, wherein at least one kernel of said BLUP model comprises a genomic relationship matrix that defines relationships based on the similarities in functional units between any two individuals in the population.
      6. A method for identifying a non-human animal organism with a desired unobserved phenotypic feature, said method comprising:
(a) obtaining genotype data for an organism against a plurality of markers (m);
(b) extracting functionally equivalent alleles for each functional unit;
(c) computing a functional unit dosage matrix (W);
(d) removing monomorphic functional units;
(e) predicting allele-substitution effects for each functional unit using a model where the vector of allele-substitution effects is drawn from a specified sampling distribution;
(f) obtaining an estimated genetic value by multiplying each allele-substitution effect for each functional unit by the corresponding vector in functional unit dosage matrix (W) and summing across functional units;
(g) utilizing said model to identify an organism having said desired unobserved phenotypic feature.
   7. The method of embodiment 6, wherein said model is linear.
   8. The method of embodiment 6, wherein said model is a Bayesian linear model.
   9. The method of embodiment 6, wherein said linear model comprises the equation set forth in Equation (2):

$$y = Xb + \sum {α}_{j SNP} {w}_{j SNP} + \sum {α}_{k FE} {w}_{k FE} + e , \qquad ( 2 )$$

wherein:
   (i) y is the phenotypic response variable,
   (ii) X is an incidence matrix that assigns the n observations in y to fixed effects,
   (iii) b is the vector of fixed effect estimates,
   (iv) α_{jSNP} is the allele substitution effect for the jth SNP and w_{j SNP} is a vector that contains the allele dosages for the jth SNP,
   (v) α_{k FE} is the allele substitution effect for the kth functional unit and w_{k SNP} is a vector that contains the allele dosages for the kth functional unit,
   (vi) Σ indicates a summation across elements, and;
   (vii) e is a vector of residuals.
      10. The method of embodiment 8 wherein the allele substitution effects follow a Gaussian distribution.
      11. The method of embodiment 8, wherein the allele substitution effects follow a scaled t distribution.
      12. The method of embodiment 8, wherein the allele substitution effects follow a two-component mixture distribution consisting of a scaled t distribution and a point mass at zero, with mixing probabilities of 1−π and π respectively.
      13. The method of embodiment 8 wherein the allele substitution effects follow a two-component mixture distribution consisting of a Gaussian distribution and a point mass at zero, with mixing probabilities of 1−π and π respectively.
      14. The method of embodiment 8, wherein the allele substitution effects follow an exponential distribution.
      15. A method for identifying an organism with a desired unobserved phenotypic feature wherein the number of functional units to be fitted to the phenotypic feature is at least one fewer than the modeled degrees of freedom, said method comprising:
(a) obtaining genotype data for an organism against a plurality of markers (m);
(b) extracting functionally equivalent alleles for each functional unit;
(c) computing a functional unit dosage matrix (W);
(d) removing monomorphic functional units;
(e) estimating allele-substitution effects for each functional unit using a linear model where the vector of allele-substitution effects is considered a fixed effect;
(f) obtaining an estimated genetic value by multiplying each allele-substitution effect for each functional unit by the corresponding vector in functional unit dosage matrix (W) and summing across functional units;
(g) utilizing said model to identify an organism having said desired unobserved phenotypic feature.
   16. The method of embodiment 15, wherein said linear model comprises the equation set forth in Equation (3):

$$y = Xb + \sum {β}_{k FE} {w}_{k FE} + e , \qquad ( 3 )$$

wherein:
   (viii) y is the phenotypic response variable,
   (ix) X is an incidence matrix that assigns the n observations in y to fixed effects,
   (x) b is the vector of fixed effect estimates,
   (xi) β_{k FE} is the vector of allele substitution effect for the kth functional unit and w_{k SNP} is a vector that contains the allele dosages for the kth functional unit,
   (xii) Σ indicates a summation across elements, and;
   (xiii) e is a vector of residuals.
      17. A method for identifying a non-human animal organism with a desired unobserved phenotypic feature, said method comprising:
(a) obtaining genotype data for an organism against a plurality of markers (m);
(b) extracting functionally equivalent alleles for each functional unit;
(c) computing a functional unit dosage matrix (W);
(d) removing monomorphic functional units;
(e) predicting a phenotypic feature with a neural network based model using functional units in W; and
(f) utilizing said neural network model to identify an organism having said desired unobserved phenotypic feature.
   18. The method of embodiment 1, 6, 15, or 17, wherein the functional unit is a gene.
   19. The method of embodiment 1, 6, 15, or 17, wherein the functional unit is a codon.
   20. The method of embodiment 1, 6, 15, or 17, wherein the functional unit is a pathway.
   21. The method of embodiment 1, 6, 15, or 17, wherein W is a loss of function dosage matrix.
   22. The method of embodiment 1, 6, 15, or 17, further comprising growing the organism.
   23. The method of embodiment 1, 6, 15, or 17, wherein the organism is an invertebrate, mammal, fish, bird, reptile, or amphibian.
   24. The method of embodiment 23, further comprising breeding said non-human animal organism to another organism.
   25. The method of embodiment 24, further comprising selecting progeny from said breeding.
   26. The method of embodiment 23, further comprising growing said non-human animal organism.
   27. A method of predicting a desired unobserved phenotypic feature for use in animal breeding, said method comprising:
(a) practicing the method of embodiment 1, 7, 15, or 17;
(b) utilizing said model to select animals having said desired unobserved phenotypic feature; and
(c) using said selected animals for further breeding.
   28. The method of embodiment 27, wherein said model is used to predict phenotypes of animals.
   29. A method for selecting a non-human animal organism with a desired unobserved phenotypic feature, said method comprising:
(a) practicing the method of embodiment 1, 7, 15, or 17;
(b) utilizing said model to select an organism having said desired unobserved phenotypic feature.
   30. The method of embodiment 29, further comprising growing the selected organism of step (b).
   31. A method of selective breeding for a desired phenotypic feature in animals, said method comprising:
(a) practicing the method of embodiment 1, 7, 15, or 17;
(b) utilizing said model to select a parental animal; and
(c) breeding the parental animal with a second animal, thereby forming a progeny animal population comprising the desired phenotypic feature.
   32. The method of embodiment 23, 27, 29, or 31, wherein the desired phenotypic feature class comprises yield, metabolism, or disease resistance.
   33. The method of embodiment 32, wherein the desired phenotypic feature class is yield, and the phenotypic feature comprises meat yield, milk yield, egg yield, or wool yield.
   34. The method of embodiment 32, wherein the desired phenotypic feature class is metabolism, and the phenotypic feature comprises fertility, feed use efficiency, or growth rate.
   35. The method of embodiment 32, wherein the desired phenotypic feature class is disease resistance, and the phenotypic feature comprises African swine fever, avian influenza, or Porcine reproductive and respiratory syndrome (PRRS).

### EXAMPLES

### Example 1

To evaluate the effectiveness of accounting for allelic heterogeneity in genomic prediction, the two-kernel GBLUP framework described above was used to predict stalk and agronomic performance traits in a temperate maize diversity panel (Hansey et al 2011, Mazaheri et al 2019). Predictions from the two-kernel GBLUP framework were compared to a single-kernel GBLUP framework in which only a genomic relationship matrix was used to model additive genetic values. Prediction accuracy was assessed using a five-fold cross-validation scheme in which the model was trained on observations for 80% of inbreds and the phenotypes were predicted for the remaining 20%. Prediction accuracies were measured using Pearson's correlation between predictive genetic values from each model and the observed phenotypes for each individual in the testing set. This process was repeated 50 times, and in each resampling run the prediction accuracies were compared. The proportion of runs in which the two-kernel/allelic heterogeneity GLUP model outperformed the conventional GBLUP model was used as a measure of significance.

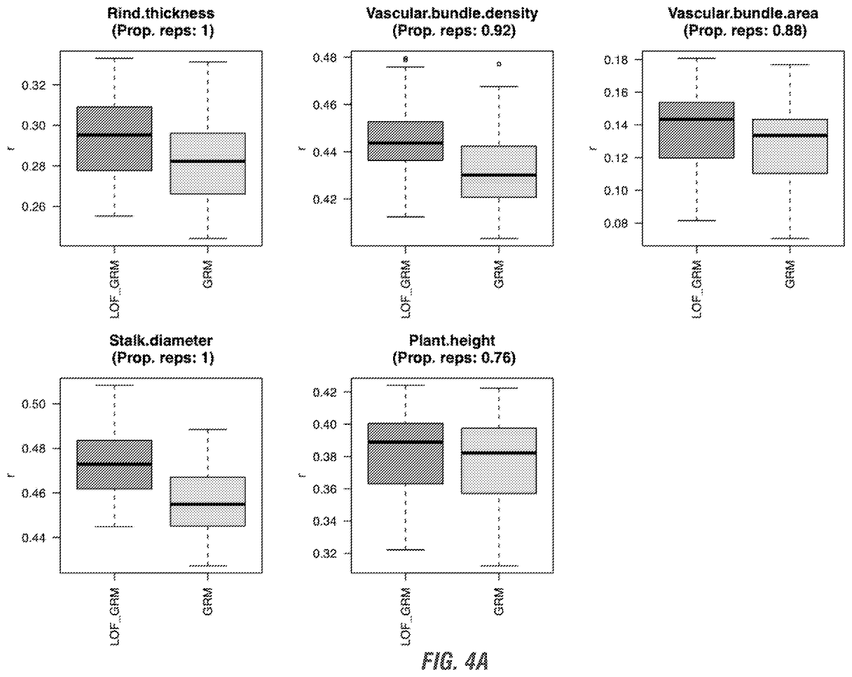

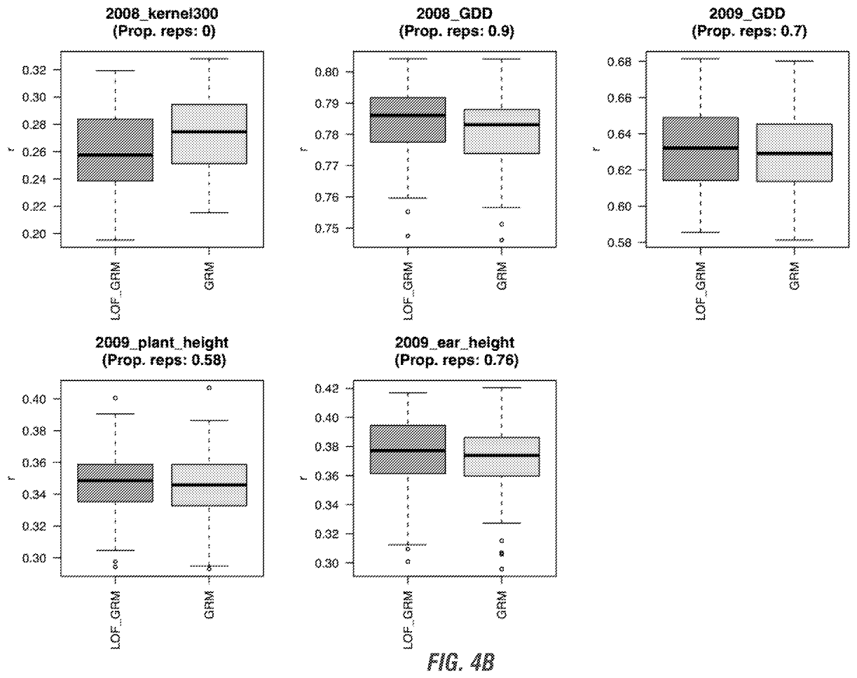

The two-kernel allelic heterogeneity model outperformed the conventional GBLUP approach for the majority of traits. Predictions for all stalk traits were higher for the two-kernel allelic heterogeneity model relative to the conventional GBLUP approach in more than 66% of the resampling runs (p<0.03284), and two traits showed higher prediction accuracies with the two-kernel allelic heterogeneity model in all resampling runs. Improvements in prediction accuracies ranged from 1.8-7.4% (FIG. 4A). A similar trend was observed for agronomic traits recorded by Hansey et al (2011). Three of the five traits analyzed showed significantly higher prediction accuracies with the two-kernel allelic heterogeneity model compared to the conventional GBLUP approach (FIG. 4B). Improvements in prediction accuracies were smaller than observed for stalk traits and ranged from −5.2-1.2%. Collectively these results support our hypothesis that considering genomic relationships based on identity by function in addition to genomic relationships derived from identity by state should improve the ability to predict complex traits.

FIG. 4 compares prediction accuracies between the two-kernel allelic heterogeneity model and a conventional GBLUP approach. The boxplots show the distribution of correlation coefficients between predicted genetic values and observed phenotypes. FIG. 4A shows the prediction accuracies for stalk traits from Mazaheri et al (2019). FIG. 4B shows the prediction accuracies for agronomic traits from Hansey et al (2011). r: Pearson's correlation coefficient; Prop. reps: Proportion of resampling replicates where the two-kernel allelic heterogeneity (LOF+GRM) model outperformed a conventional GBLUP approach (GRM); 2008_kernel300: 300 kernel weight measured in 2008 field season; 2008_GDD: growing degree days to anthesis in 2008 field season.

The following are prophetic examples:

### Example 2

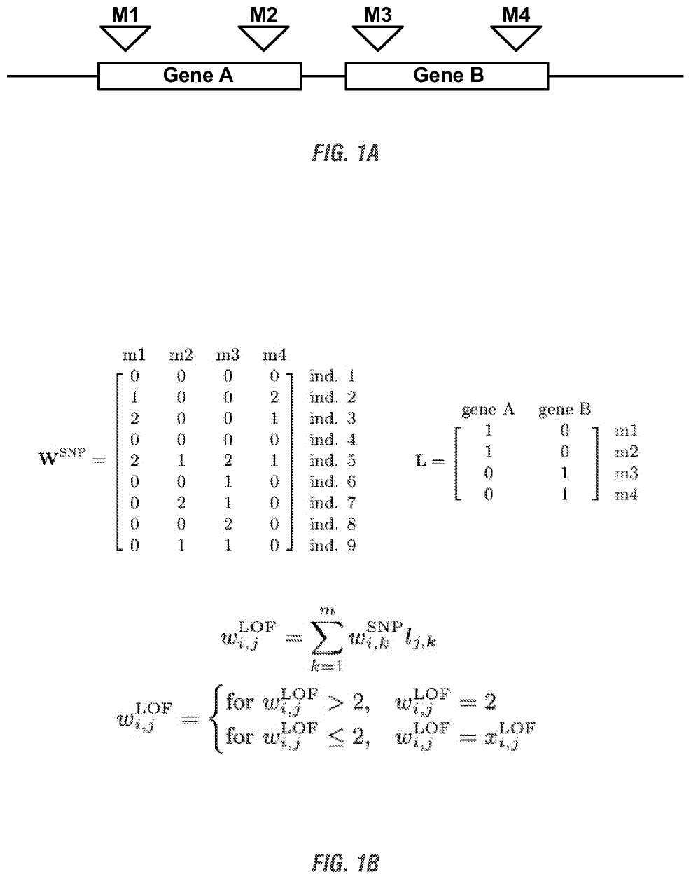

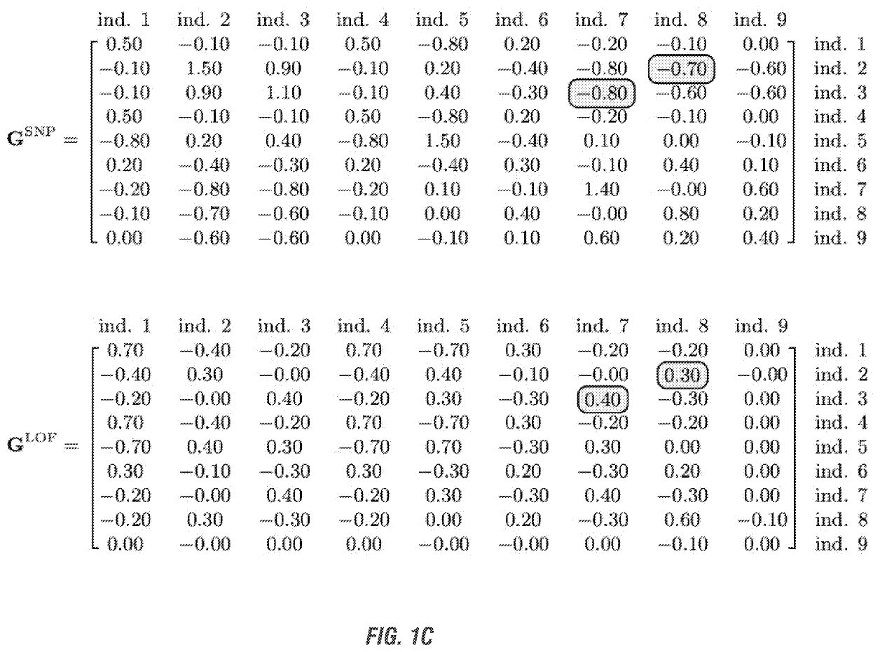

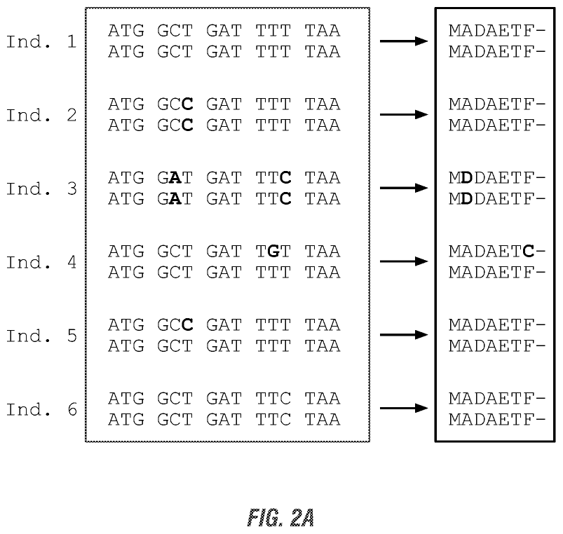

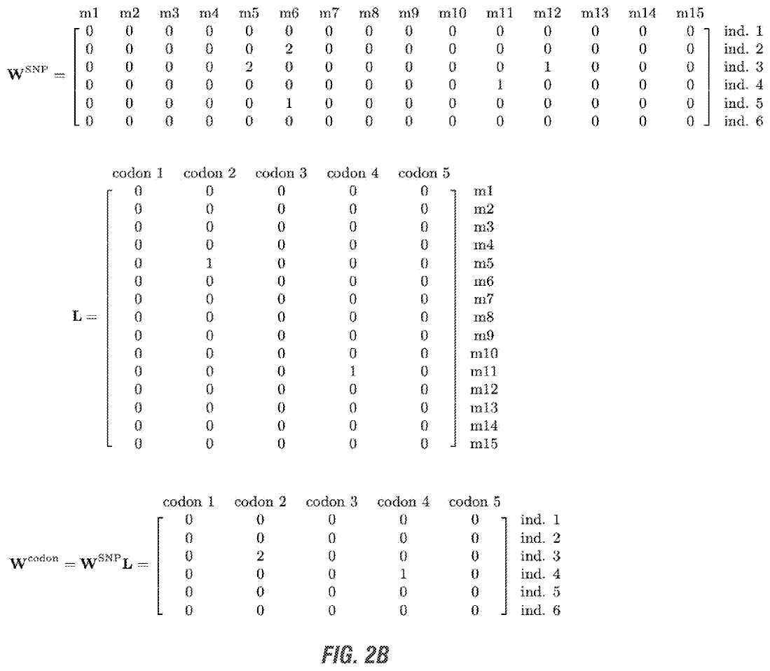

Codon as functional unit: The genetic code specifies a set of rules which dictate the sequence of amino acids that are to be translated by a set of triplets of DNA nucleotides. There are 64 unique DNA triplets, 61 of which code for the 20 amino acids while the remaining three represent stop signals that cause translation to cease. Given that there are more than three times as many codons as amino acids, there is some redundancy in the genetic code; thus, functionally equivalent alleles—i.e., synonymous substitutions—can be collapsed at a codon level and can be used to estimate relatedness based on functional similarities using a similar framework described above. This approach is outlined in FIG. 2.
a. W^{SNP} as above is a matrix of allele dosages for each SNP for every individual in the population; L is an incidence matrix that assigns non-synonymous SNP variants to codons; and a matrix of counts of functionally equivalent codons can be computed by taking the cross product of W^{SNP} and L. Genomic relationships based on functional similarities at the codon level can be computed following the methods outlined for LoF genes.

### Example 3

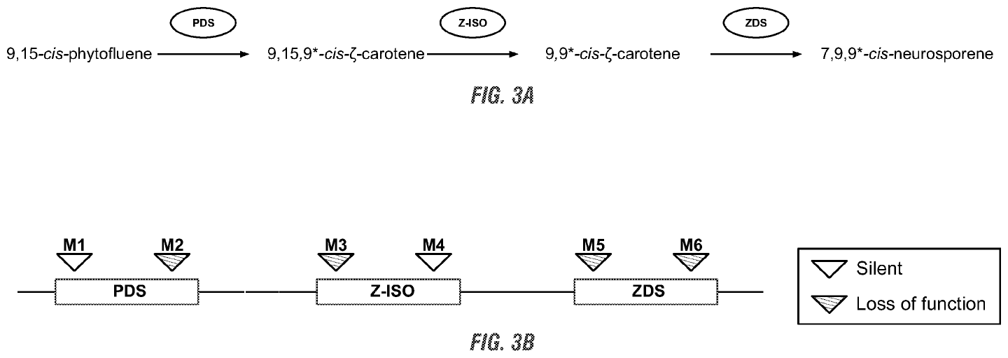

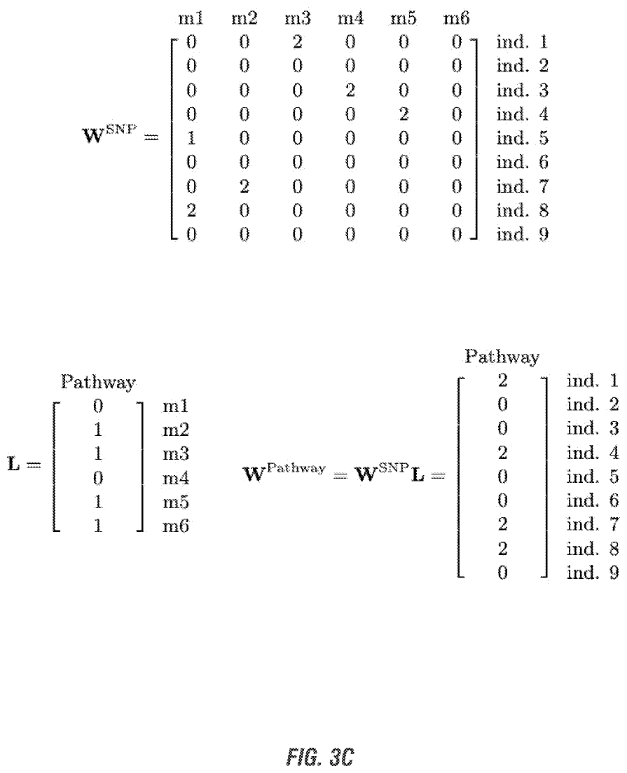

Pathway as function unit: Pathways can also be considered as another functional unit in which functionally equivalent alleles can be aggregated. This is demonstrated in FIG. 3 using a hypothetical example of the carotenoid pathway in maize (Wurtzel, Cuttriss and Vallabhaneni, 2012; https://www.frontiersin.org/articles/10.3389/fpls.2012.00029/fill). Here, a set of four loss of function variants (M2, M3, M4, M5, and M6) have been genotyped in nine individuals in the population. The L matrix assigns loss of function variants to the pathway and the cross product of W, allele dosages for LoF sites, and L give LoF dosages for the carotenoid pathway. Genomic relationships based on functional similarities at the pathway level can be computed following the methods outlined for LoF genes.

What is claimed is:

1. A method for identifying a non-human animal organism with a desired unobserved phenotypic feature, said method comprising:
(a) obtaining genotype data for an organism against a plurality of markers (m);
(b) extracting functionally equivalent alleles for each functional unit;
(c) computing a functional unit dosage matrix (W);
(d) removing monomorphic functional units;
(e) computing an identity by function relationship matrix;
(f) predicting said unobserved phenotypic feature using a best linear unbiased prediction (BLUP) model; and
(g) utilizing said model to identify a non-human animal organism having said desired unobserved phenotypic feature.

2. The method of claim 1, wherein said BLUP model is a two-kernel BLUP model.

3. The method of claim 1, wherein said BLUP model comprises the equation set forth in Equation (1):
$$y = Xb + {Zu}_{GRM} + {Zu}_{FE} + e , \qquad ( 1 )$$
wherein:
(viii) y is the phenotypic response variable,
(ix) X is an incidence matrix that assigns the n observations in y to fixed effects,
(x) b is the vector of fixed effect estimates,
(xi) Z is an incidence matrix that assigns observations to random additive genetic values contained in u_{GRM} and u_{FE},
(xii) u_{GRM} is a vector of additive genetic values modeled by identity by descent relationships,
(xiii) u_{FE} is a vector of additive genetic values modeled by identity by function relationships (sharing of functionally equivalent functional units), and;
(xiv) e is a vector of residuals.

4. The method of claim 3, wherein the random effects follow a Gaussian distribution.

5. The method of claim 1, wherein at least one kernel of said BLUP model comprises a genomic relationship matrix that defines relationships based on the similarities in functional units between any two individuals in the population.

6. A method for identifying a non-human animal organism with a desired unobserved phenotypic feature, said method comprising:
(a) obtaining genotype data for an organism against a plurality of markers (m);
(b) extracting functionally equivalent alleles for each functional unit;
(c) computing a functional unit dosage matrix (W);
(d) removing monomorphic functional units;
(e) predicting allele-substitution effects for each functional unit using a model where the vector of allele-substitution effects is drawn from a specified sampling distribution;
(f) obtaining an estimated genetic value by multiplying each allele-substitution effect for each functional unit by the corresponding vector in functional unit dosage matrix (W) and summing across functional units;
(g) utilizing said model to identify an organism having said desired unobserved phenotypic feature.

7. The method of claim 6, wherein said model is linear.

8. The method of claim 6, wherein said model is a Bayesian linear model.

9. The method of claim 6, wherein said linear model comprises the equation set forth in Equation (2):
$$y = Xb + \sum {α}_{j SNP} {w}_{j SNP} + \sum {α}_{k FE} {w}_{k FE} + e , \qquad ( 2 )$$
wherein:
(xiv) y is the phenotypic response variable,
(xv) X is an incidence matrix that assigns the n observations in y to fixed effects,
(xvi) b is the vector of fixed effect estimates,
(xvii) α_{jSNP} is the allele substitution effect for the jth SNP and w_{j SNP} is a vector that contains the allele dosages for the jth SNP,
(xviii) α_{k FE} is allele substitution effect for the kth functional unit and w_{j SNP} is a vector that contains the allele dosages for the kth functional unit,
(xix) Σ indicates a summation across elements, and;
(xx) e is a vector of residuals.

10. The method of claim 8 wherein the allele substitution effects follow a Gaussian distribution.

11. The method of claim 8, wherein the allele substitution effects follow a scaled t distribution.

12. The method of claim 8, wherein the allele substitution effects follow a two-component mixture distribution consisting of a scaled t distribution and a point mass at zero, with mixing probabilities of 1−π and π respectively.

13. The method of claim 8 wherein the allele substitution effects follow a two-component mixture distribution consisting of a Gaussian distribution and a point mass at zero, with mixing probabilities of 1−π and π respectively.

14. The method of claim 8, wherein the allele substitution effects follow an exponential distribution.

15. A method for identifying an organism with a desired unobserved phenotypic feature wherein the number of functional units to be fitted to the phenotypic feature is at least one fewer than the modeled degrees of freedom, said method comprising:
(a) obtaining genotype data for an organism against a plurality of markers (m);
(b) extracting functionally equivalent alleles for each functional unit;
(c) computing a functional unit dosage matrix (W);
(d) removing monomorphic functional units;
(e) estimating allele-substitution effects for each functional unit using a linear model where the vector of allele-substitution effects is considered a fixed effect;
(f) obtaining an estimated genetic value by multiplying each allele-substitution effect for each functional unit by the corresponding vector in functional unit dosage matrix (W) and summing across functional units;
(g) utilizing said model to identify an organism having said desired unobserved phenotypic feature.

16. The method of claim 15, wherein said linear model comprises the equation set forth in Equation (3):
$$y = Xb + \sum {β}_{k FE} {w}_{k FE} + e , \qquad ( 3 )$$
wherein:
(xxi) y is the phenotypic response variable,
(xxii) X is an incidence matrix that assigns the n observations in y to fixed effects,
(xxiii) b is the vector of fixed effect estimates,
(xxiv) β_{k FE} is the vector of allele substitution effect for the kth functional unit and w_{k SNP} is a vector that contains the allele dosages for the kth functional unit,
(xxv) Σ indicates a summation across elements, and;
(xxvi) e is a vector of residuals.

17. A method for identifying a non-human animal organism with a desired unobserved phenotypic feature, said method comprising:
(a) obtaining genotype data for an organism against a plurality of markers (m);
(b) extracting functionally equivalent alleles for each functional unit;
(c) computing a functional unit dosage matrix (W);
(d) removing monomorphic functional units;
(e) predicting a phenotypic feature with a neural network based model using functional units in W; and
(f) utilizing said neural network model to identify an organism having said desired unobserved phenotypic feature.

18. The method of claim 1, 6, 15, or 17, wherein the functional unit is a gene.

19. The method of claim 1, 6, 15, or 17, wherein the functional unit is a codon.

20. The method of claim 1, 6, 15, or 17, wherein the functional unit is a pathway.

21. The method of claim 1, 6, 15, or 17, wherein W is a loss of function dosage matrix.

22. The method of claim 1, 6, 15, or 17, further comprising growing the organism.

23. The method of claim 1, 6, 15, or 17, wherein the organism is an invertebrate, mammal, fish, bird, reptile, or amphibian.

24. The method of claim 23, further comprising breeding said non-human animal organism to another organism.

25. The method of claim 24, further comprising selecting progeny from said breeding.

26. The method of claim 23, further comprising growing said non-human animal organism.

27. A method of predicting a desired unobserved phenotypic feature for use in animal breeding, said method comprising:
(a) practicing the method of claim 1, 7, 15, or 17;
(b) utilizing said model to select animals having said desired unobserved phenotypic feature; and
(c) using said selected animals for further breeding.

28. The method of claim 27, wherein said model is used to predict phenotypes of animals.

29. A method for selecting a non-human animal organism with a desired unobserved phenotypic feature, said method comprising:
(a) practicing the method of claim 1, 7, 15, or 17;
(b) utilizing said model to select an organism having said desired unobserved phenotypic feature.

30. The method of claim 29, further comprising growing the selected organism of step (b).

31. A method of selective breeding for a desired phenotypic feature in animals, said method comprising:
(a) practicing the method of claim 1, 7, 15, or 17;
(b) utilizing said model to select a parental animal; and
(c) breeding the parental animal with a second animal, thereby forming a progeny animal population comprising the desired phenotypic feature.

32. The method of claim 23, wherein the desired phenotypic feature class comprises yield, metabolism, or disease resistance.

33. The method of claim 27, wherein the desired phenotypic feature class comprises yield, metabolism, or disease resistance.

34. The method of claim 29, wherein the desired phenotypic feature class comprises yield, metabolism, or disease resistance.

35. The method of claim 31, wherein the desired phenotypic feature class comprises yield, metabolism, or disease resistance.

36. The method of claim 32, wherein the desired phenotypic feature class is yield, and the phenotypic feature comprises meat yield, milk yield, egg yield, or wool yield.

37. The method of claim 33, wherein the desired phenotypic feature class is yield, and the phenotypic feature comprises meat yield, milk yield, egg yield, or wool yield.

38. The method of claim 34, wherein the desired phenotypic feature class is yield, and the phenotypic feature comprises meat yield, milk yield, egg yield, or wool yield.

39. The method of claim 35, wherein the desired phenotypic feature class is yield, and the phenotypic feature comprises meat yield, milk yield, egg yield, or wool yield.

40. The method of claim 32, wherein the desired phenotypic feature class is metabolism, and the phenotypic feature comprises fertility, feed use efficiency, or growth rate.

41. The method of claim 33, wherein the desired phenotypic feature class is metabolism, and the phenotypic feature comprises fertility, feed use efficiency, or growth rate.

42. The method of claim 34, wherein the desired phenotypic feature class is metabolism, and the phenotypic feature comprises fertility, feed use efficiency, or growth rate.

43. The method of claim 35, wherein the desired phenotypic feature class is metabolism, and the phenotypic feature comprises fertility, feed use efficiency, or growth rate.

44. The method of claim 32, wherein the desired phenotypic feature class is disease resistance, and the phenotypic feature comprises African swine fever, avian influenza, or Porcine reproductive and respiratory syndrome (PRRS).

45. The method of claim 33, wherein the desired phenotypic feature class is disease resistance, and the phenotypic feature comprises African swine fever, avian influenza, or Porcine reproductive and respiratory syndrome (PRRS).

46. The method of claim 34, wherein the desired phenotypic feature class is disease resistance, and the phenotypic feature comprises African swine fever, avian influenza, or Porcine reproductive and respiratory syndrome (PRRS).

47. The method of claim 35, wherein the desired phenotypic feature class is disease resistance, and the phenotypic feature comprises African swine fever, avian influenza, or Porcine reproductive and respiratory syndrome (PRRS).
